(19) United States
(12) Patent Application Publication
(10) Pub. No.: US 2016/0184077 A1
(43) Pub. Date: Jun. 30, 2016

(54) Circular Sectional Inserts For Insertion with a Trunk Section in Endoprosthesis for Aortic Aneurysm

(71) Applicant: Cordis Corporation, Fremont, CA (US)

(72) Inventors: Animesh CHOUBEY, Fremont, CA (US); David MAJERCAK, Livermore, CA (US)

(73) Assignee: Cordis Corporation, Fremont, CA (US)

(21) Appl. No.: 14/587,621

(22) Filed: Dec. 31, 2014

(51) Int. Cl. A61F 2/07

(52) U.S. Cl. CPC A61F 2/07; A61F 2250/001; A61F 2002/075; A61F 2002/065

(57) ABSTRACT

Described are various embodiments of an improved endoprosthesis that includes at least one tubular graft section coupled to additional tubular graft sections which are then coupled to a tubular bifurcated main section. Various embodiments described and shown herein allow for a health care provider to design and select an appropriate AAA implant for AAA presentations other than an infrarenal AAA. The implants can also be utilized for other arterial aneurysms.

### BACKGROUND

An aneurysm is an abnormal dilation of a layer or layers of an arterial wall, usually caused by a structural defect due to hardening of the artery walls or other systemic defects such as aortic dissection due to high blood pressure. The widely accepted approach to treating an aneurysm in the abdominal aorta (i.e., an “abdominal aortic aneurysm” or “AAA”) is by surgical repair, involving replacing the aneurysmal segment with a prosthetic device. This surgery is a major undertaking, with associated high risks and with significant mortality and morbidity.

A typical surgical repair for AAA is performed by making an incision into the abdomen to allow the physician to access the aorta (FIG. 8A). Once the aorta is accessible, it may be clamped to allow the surgeon to cut open the aorta and suture one graft end proximal to the heart. The other end of the graft is sutured to the aorta at a location past the aneurysm. This allows the blood flow from the heart to bypass the weakened area of the aorta.

One alternative to the surgical repair is to use an endovascular procedure, i.e., catheter directed, techniques for the treatment of aneurysms, specifically for AAA. This has been facilitated by the development of vascular stents, which can and have been used in conjunction with standard or thin-wall graft material in order to create a stent-graft or endograft. The potential advantages of less invasive treatments have included reduced surgical morbidity and mortality along with shorter hospital and intensive care unit stays.

One concern with the use of an endograft (or endoprosthesis) for AAA is that most if not all AAA endoprosthesis are configured for presentation of AAA as an infrarenal AAA. As shown in FIG. 8AI, an infrarenal typically presents sufficient landing zones for the implant to achieve a tight seal between the inner surface of the vessel wall of the aorta and the outer surface of the endoprosthesis. Where the distance between the renal arteries and aneurysm (i.e., the “neck length”) is less than 15 mm, it is believed that complications may result from the use of an endoprosthesis designed for an infrarenal presentation. Thus, in the presentation of a neck length of less than 15 mm, a juxtarenal AAA (FIG. 8BII), pararenal AAA (FIG. 8BIII), or a suprarenal AAA (FIG. 8BIV), it is believed that complications would certainly result from the use of the existing AAA endoprosthesis.

Others in this field have attempted to overcome the drawbacks of existing AAA endoprosthesis by utilizing what is known in the field as the “fenestrated technique”. This technique relies on hand-made customized fenestrations to incorporate both the renal and superior mesenteric arteries into such bespoke endoprosthesis for juxtarenal to suprarenal AAAs. In one aspect of the fenestrated technique, a physician can make openings or fenestrations by hand to an off-the-shelf AAA implant. The drawbacks to physician modified fenestrated implants are that the implants are not FDA approved, requiring the physician to apply for a regulatory waiver and such fenestrated implants may take hours to make by the physician. To alleviate these drawbacks, manufacturers have provided customized fenestrated implant based on imaging of the aneurysm 6-12 weeks before the scheduled implant. However, one drawback to this technique is that a peculiar anatomy of the renal arteries may render the customized implant ineffective. For example, there may be an extra renal or hepatic artery involved, as well as renal arteries that are oriented upward. Additionally, the bespoke implants typically require a long-lead time by which time the anatomy of the AAA could have changed significantly resulting in branching arteries that do not align with the fenestrations. Even if the known implant could be modified during the day of the implant by the physician (to avoid the time lag issue for the customized implant noted earlier), such physician-modified-implant (as well as the custom-made implant) may still not be ideal due to angulation of the anatomy causing the custom fenestrations to shift from the ideal alignment with the branching arteries.

### SUMMARY OF THE DISCLOSURE

Accordingly, we have devised an implantable endoprosthesis overcomes the disadvantages in the bespoke fenestration in that a physician does not have to hand make a custom implant a few hours before the implantation procedure. And our invention overcomes the problems associated with an implant made by order weeks in advance before the actual AAA operation whereby the anatomy or the aneurysm may have changed during the time the implant was ordered and actually implanted. In brief, the invention provides for three key improvements: (1) ease of use in the simplification of deployment for one fenestration at a time; (2) in-situ alignment of each opening to the targeted branching artery resulting in improved clinical outcomes; and (3) the overall profile of the endoprosthesis is ultra-low (i.e., less than 16F for large native artery and in most cases, less than 12 French) because each portion of the endoprosthesis is smaller while requiring only one extra guidewire lumen.

In particular, we have devised an implantable endoprosthesis that includes two main portions. The first portion includes a graft material defining a generally tubular graft extending from a first portion inlet opening to first portion outlet opening. The first portion includes a first peripheral opening formed on a peripheral surface of the generally tubular graft to allow the first peripheral opening to communicate with the inlet and outlet of the first portion. The second portion extends along the longitudinal axis. The second portion includes a graft material defining a second portion inlet opening to a trunk section that extends along the longitudinal axis to a bifurcation section having two limbs with respective limb outlet openings. The second portion has a retention member coupled via a hoop structure to the inlet opening and two spaced apart peripheral openings formed through the peripheral surface of the trunk section to allow the spaced apart second portion's peripheral openings to communicate with the trunk inlet and limb outlets such that radial alignment of the first peripheral opening with the second peripheral openings allows fluid communication through the first peripheral opening and the second peripheral opening.

In addition to the embodiments described above, other features recited below can be utilized in conjunction therewith. For example, each of the first and second portions comprises a plurality of stent hoops spaced apart from each other along the longitudinal axis and attached to a graft material to define a stent graft composite implant, each of the stent hoops having a sinusoidal configuration disposed about the longitudinal axis with apices spaced apart along the longitudinal axis; one apex of one stent hoop is disposed between two apices of another stent hoop; the generally tubular graft comprises a material selected from a group consisting of nylon, ePTFE, PTFE, Dacron and combinations thereof; the plurality of stent hoops are disposed on a peripheral inside surface of the stent-graft; a first peripheral opening is formed through the graft material about the longitudinal axis of the first portion proximate the first end so that the first peripheral opening communicates with a mesenteric artery when the first and second implants are deployed together in an abdominal artery; a second peripheral opening is formed through the graft material about the longitudinal axis of the first portion so that the second peripheral opening and a pair of peripheral openings (e.g., openings 104+128 or 106+130) communicates with a renal artery when the implant is deployed in the abdominal artery to allow fluid communication from the renal artery to the second; a third peripheral opening is formed through the graft material about the longitudinal axis of the second portion so that the third peripheral opening communicates with another renal artery when the implant is deployed in the abdominal artery to allow fluid communication from the renal artery to the third peripheral opening; the first portion is radially adjustable with respect to the second portion so that the first peripheral opening on the first portion is generally aligned to the first peripheral opening on the second portion; an arterial stent graft extension having a graft material in a generally tubular configuration with a generally circular opening at one end tapering towards a smaller second generally circular extension opening proximate another end, the arterial stent graft extension being configured for insertion into at least one of the peripheral openings of the first and second portions; at least one stent hoop expandable to support the arterial stent graft; or a stent graft tubular extension is provided for insertion into each of the two limbs to allow for fluid flow from the first opening of the first portion through the second and third portions and to the respective limbs and out through each of the extensions.

### BRIEF DESCRIPTION OF THE FIGURES

The foregoing and other features and advantages of the invention will be apparent from the following, more particular description of preferred embodiments of the invention, as illustrated in the accompanying drawings.

FIG. 1 illustrates a first main section 108 and a second main section 108 of the implant 100;

FIG. 1A illustrates via a close-up of the peripheral opening 104 specially configured for graft portion 103 so that the opening 104 can be dilated to match the actual branching artery when in-situ;

FIG. 2 illustrates the limb extensions for the limbs of the trunk section 108;

FIG. 3 illustrates arterial graft extension for the peripheral openings 104 and 130 of the implant 100;

FIG. 4A illustrates in a perspective view of both sections 102 and 108 in the AAA presented as a juxtarenal AAA;

FIG. 4B illustrates in a perspective view of another variation of implant 100, indicated as 100′ in which the multiple first sections 102 can be used for a suprarenal AAA;

FIGS. 5-7 illustrate perspective views of yet another variation of implant 100, indicated here as 100″ where a scalloped first portion 102″ can be utilized with the main section 108″;

FIGS. 7A and 7B illustrate a delivery device for the implants described herein;

FIG. 8A illustrates a human abdominal aorta with the usual arteries branching therefrom;

FIG. 8BI illustrates a presentation of an infrarenal AAA;

FIG. 8BII illustrates a presentation of a juxtarenal AAA;

FIG. 8BIII illustrates a presentation of a pararenal AAA; and

FIG. 8BIV illustrates a presentation of a suprarenal AAA;

The accompanying drawings, which are incorporated herein and constitute part of this specification, illustrate presently preferred embodiments of the invention, and, together with the general description given above and the detailed description given below, serve to explain features of the invention (wherein like numerals represent like elements.

### MODES OF CARRYING OUT THE INVENTION

The following detailed description should be read with reference to the drawings, in which similar or identical elements in different drawings are identically numbered. The drawings, which are not necessarily to scale, depict selected embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±50% of the recited value, e.g. “about 50%” may refer to the range of values from 51% to 99%. In addition, as used herein, the terms “patient,” “host,” “user,” and “subject” refer to any human or animal subject and are not intended to limit the systems or methods to human use, although use of the subject invention in a human patient represents a preferred embodiment. The uses of the terms “cranial” or “caudal” are in this application are used to indicate a relative position or direction with respect to the person receiving the implant. As applied to “cranial,” the term indicates a position or direction closer to the heart, while the term “caudal” indicates a position or direction further away from the heart of such a subject.

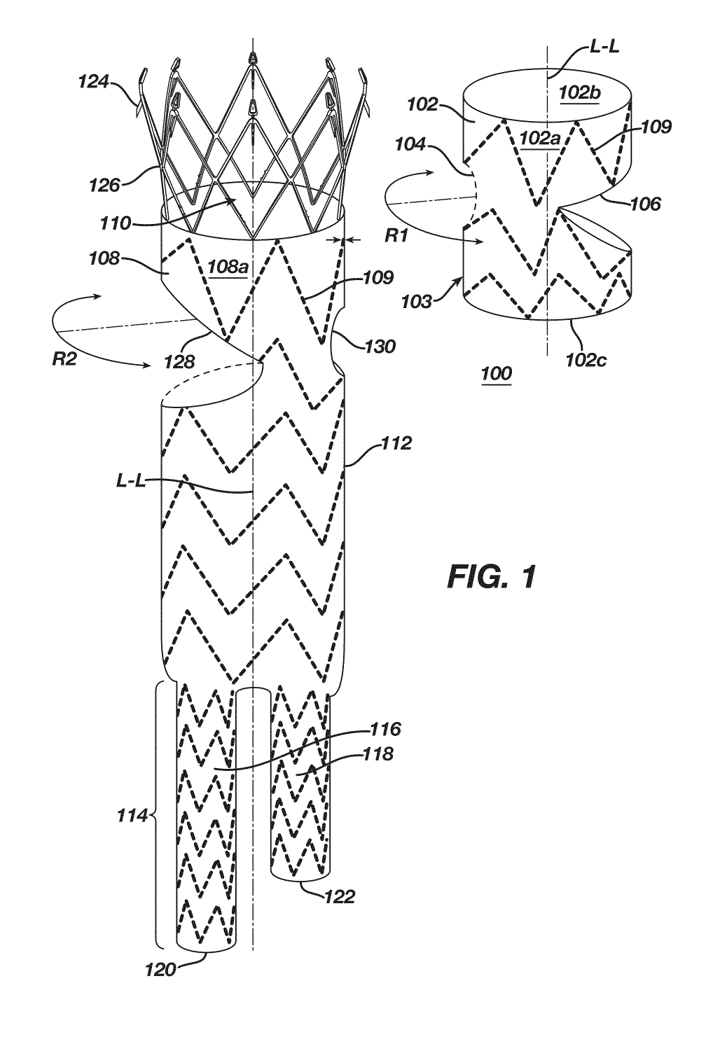

A first embodiment of an endovascular implant 100 is shown in FIG. 1 that can be used with limb extensions in EVAR procedures for AAAs that is other than infra-renal. In other words, the implant 100 can be used in AAA that categorized as juxtarenal, pararenal or suprarenal type AAAs due to its particular configuration. In particular, as shown in FIG. 1, a first portion 102 (of the implant 100) is configured to extend along a longitudinal axis L-L. The first portion 102 may be made from a suitable bio-compatible graft material 102a such as, for example, a material selected from a group consisting of nylon, ePTFE, PTFE, Dacron and combinations thereof.

The graft material 102a of the first portion 102 defines a generally tubular graft 103 that extends from a first portion's inlet opening 102b to first portion outlet opening 102c. The first portion 102 includes a first peripheral opening 104 formed on (and through) a peripheral surface of the generally tubular graft 103 to allow the first peripheral opening 104 to communicate with the inlet 102b and outlet 102c of the first portion 102.

The implant 100 also includes a second portion 108. The second portion 108 extends along the longitudinal axis L-L and may include a graft material 108a, which can be selected from a suitable biocompatible material as noted earlier with respect to material 102a of the first portion 102. The graft material 108a, by virtue of its design configuration, defines a second portion inlet opening 110 of a trunk section 112 that extends along the longitudinal axis L-L to a bifurcation section 114. The bifurcation 114 has two limbs 116, 118 with respective limb outlet openings 120, 122. Note that the second portion 108 has a retention member 124 designed to be coupled (via a stent or hoop structure 126 to the inlet opening 110. Similar to the first portion 102, two spaced apart peripheral openings 128, 130 are formed through the peripheral surface of the trunk section 112. This allows the spaced apart peripheral openings 128, 130 (of the second portion 108) to communicate with the trunk inlet 110 and limb outlets 120, 122 such that a radial alignment of the first peripheral opening 104 (of the first portion 102) to the second peripheral openings 128, 130 (of the second portion 108) is achieved. In other words, the configuration of the two portions (102 and 108) along with its respective peripheral openings (104, 106, 128 and 130) allows for fluid communication from the inlet 102b of the first portion 102 through its first peripheral opening 104 and the second peripheral opening 128 of the second portion 108.

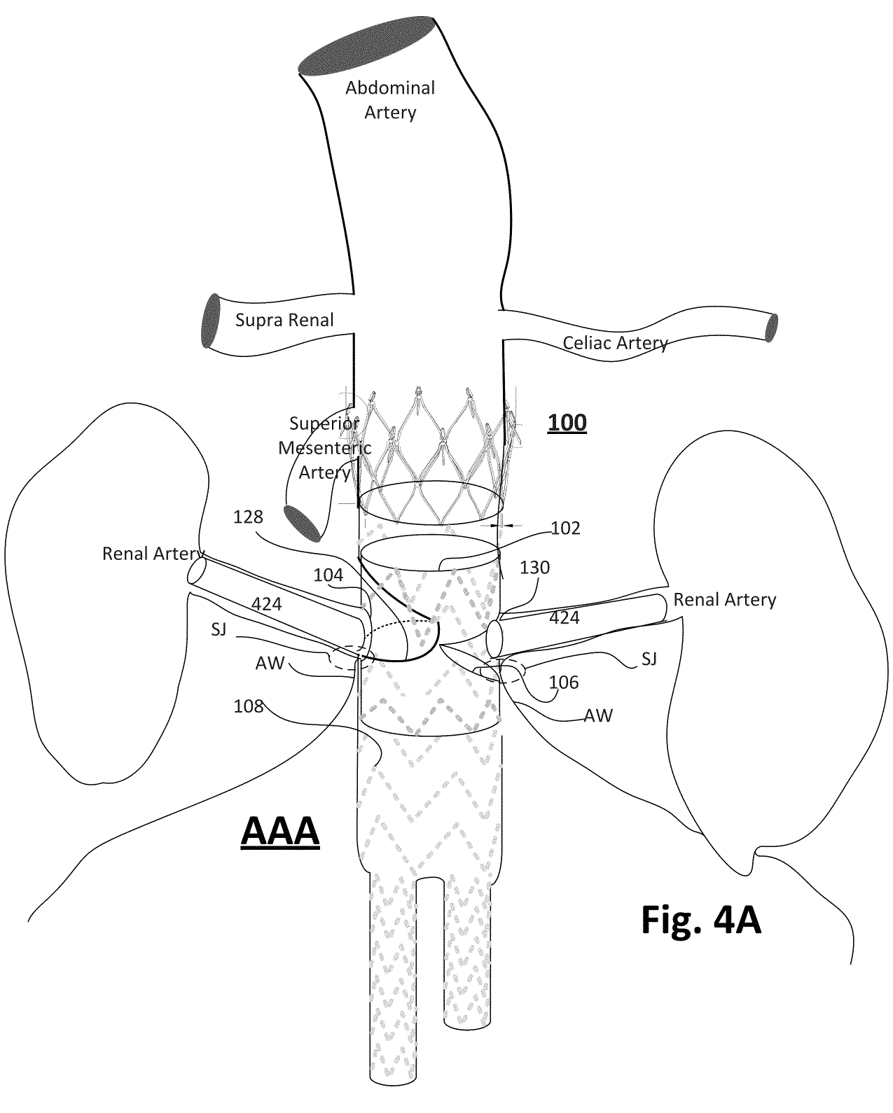

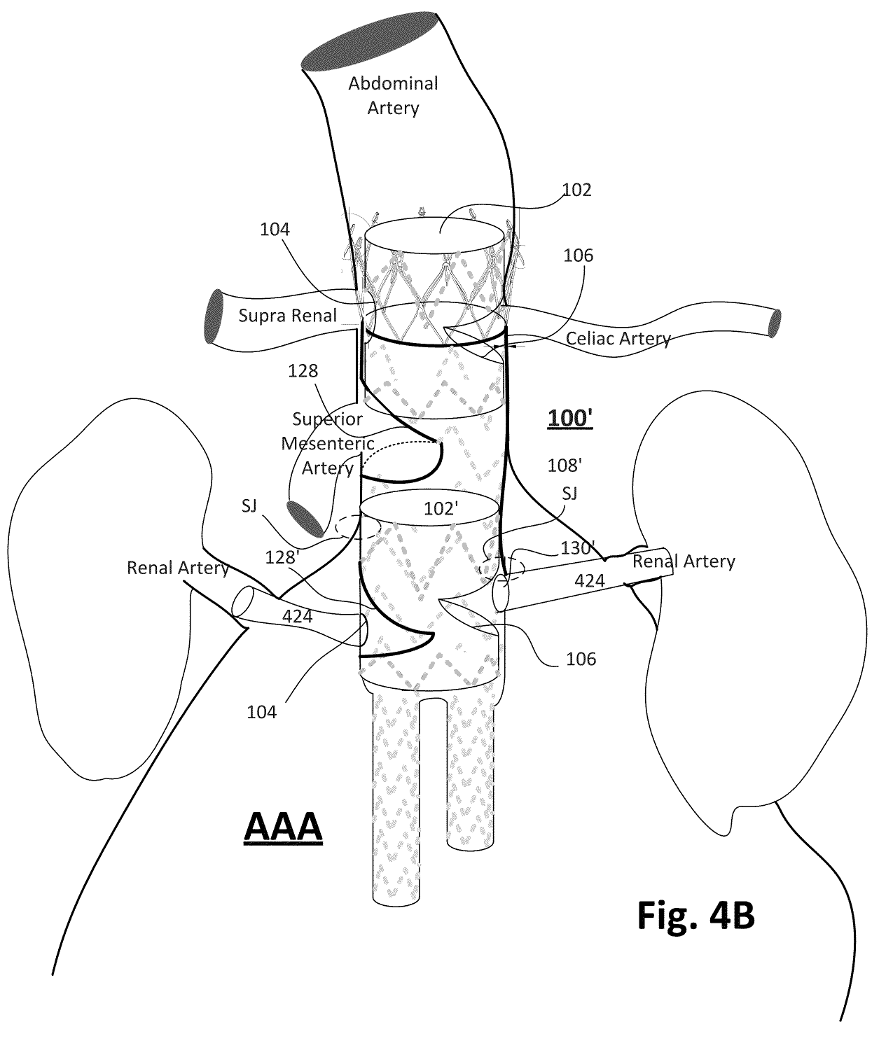

In one exemplary application, shown here in FIG. 4A, second peripheral opening 106 is formed through the graft material 102a of the first portion 102 about the longitudinal axis L-L of the first portion 102 so that the counterpart peripheral openings 104 and 106 of the first portion 102 are aligned with the respective second peripheral openings 128, 130 of the second portion 108 and a pair of peripheral openings (104+128 as one pair and 106+130 as the other pair) communicates with a renal artery when the implant 100 is deployed in the abdominal artery.

A variation of the implant 100, denoted as 100′, can be seen in FIG. 4B. In FIG. 4B, the implant 100′ is configured such that the first peripheral opening (104) of the first portion is formed through the graft material (102a) about the longitudinal axis (L-L) of the first portion (102) proximate the first end (102b) so that the first peripheral opening (104) communicates with a mesenteric artery when the first and second implants are deployed together in an abdominal artery.

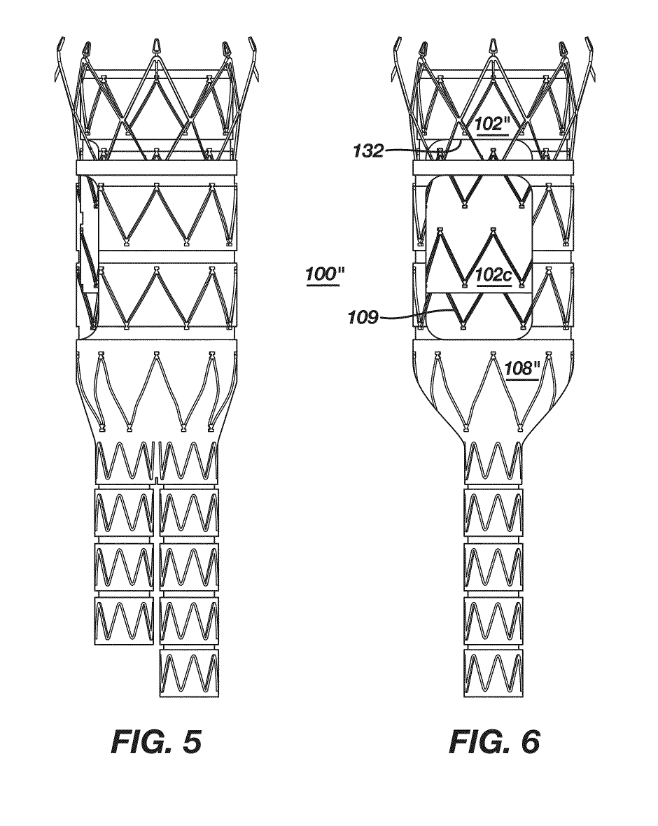

In another embodiment, shown here in FIGS. 5 and 6, the first peripheral opening 106 of the first portion 102 can be configured as a scallop cut-out 132 that extend from a periphery of the first portion 102 to the outlet opening 102c of the first portion 102. That is, the cut-out 132 has three sides instead of four sides, as in the embodiment of FIG. 1.

Referring to FIG. 3, an arterial stent graft extension (or bridging stent, as known in the field) 424 can be used for insertion into peripheral openings 104 and 130 so that side arteries can be incorporated into the flow of the implant. The extension 424 has a suitable biocompatible graft material 424a similar to the graft material of the main portions noted earlier. The extension 424 is configured as a generally tubular flow through structure. In one embodiment, the extension 424 has a generally circular opening 424b at one end 425a. The extension 424 tapers from the first end 425a towards a smaller second generally circular extension opening 424c proximate the other end 425b. The arterial stent graft extension 424 is configured for insertion into at least one of the peripheral openings of the first and second portions with retainers provided proximate each end 425a and 425b to retain the extension to the main portions of the implant or the blood vessel.

Where a self-supporting structure is required for each of the first and second portions 102 and 108 or the limb extensions 130, a plurality of stent hoops can be attached to the graft material of the implant. In particular, each of the first and second portions 102, 108 may have a plurality of stent hoops 109 spaced apart from each other along the longitudinal axis L-L and attached to the graft material to define the preferred composite implant. It is noted that each of the stent hoops 109 has a sinusoidal configuration disposed about the longitudinal axis L-L with apices spaced apart along the longitudinal axis L-L. To allow for a thin profile prior to deployment, i.e., less than 12 French (and in some cases where the native artery is large, the implant pre-deployment profile can be less than 16 French), we have devised the stent hoop so that one apex of one stent hoop is disposed between two apices of another stent hoop shown as an example in FIG. 1.

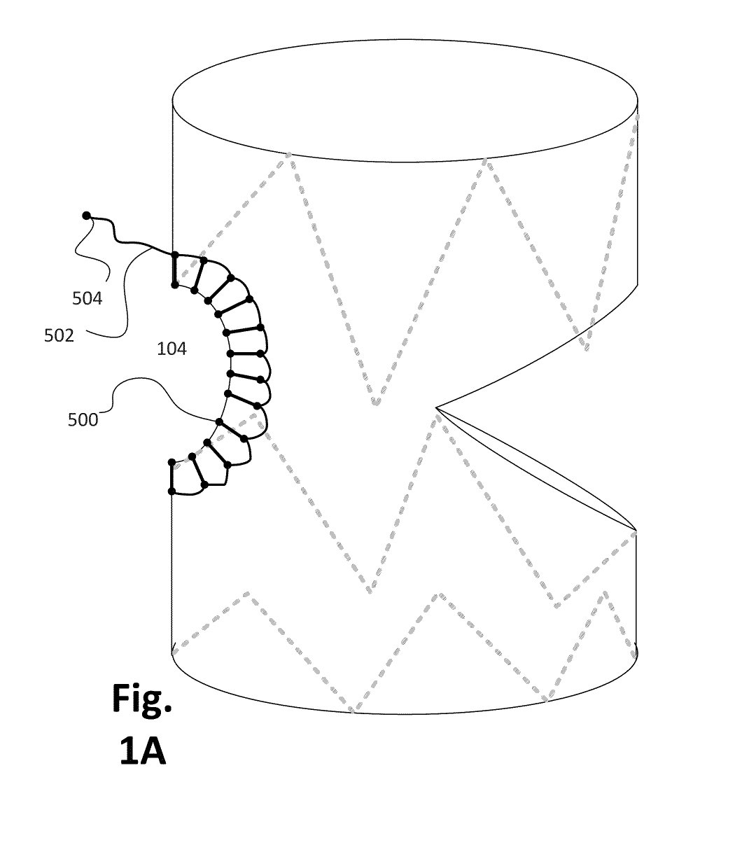

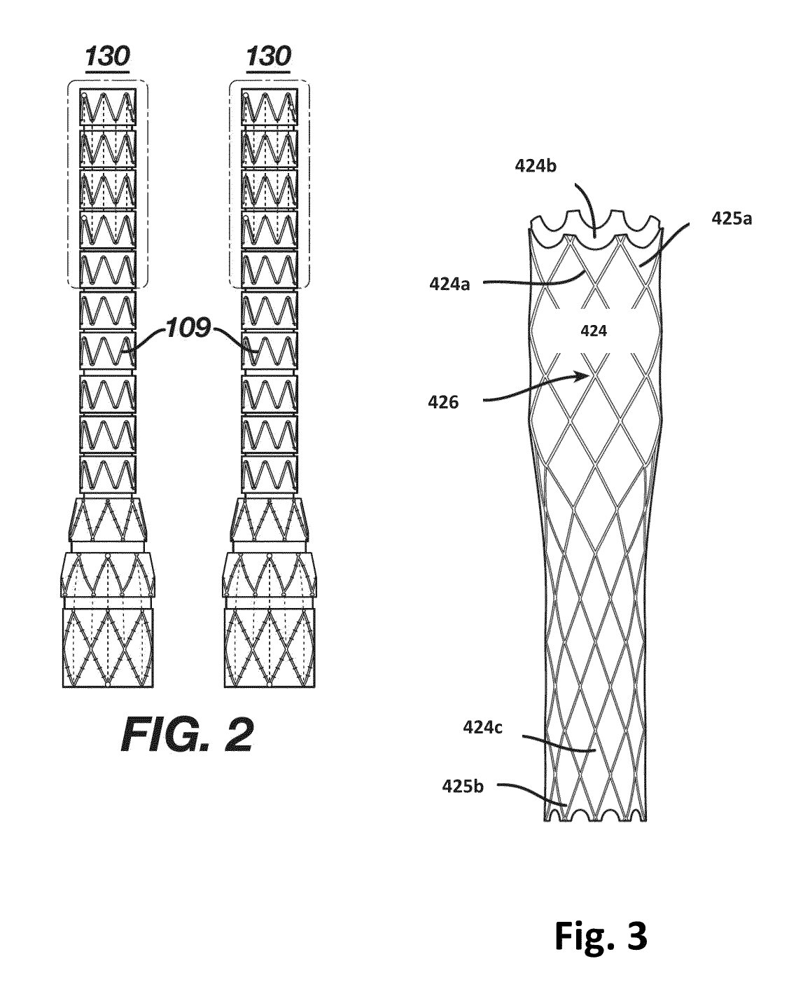

In the preferred embodiment, the plurality of stent hoops 109 are disposed on a peripheral inside surface of the stent-graft first portion 102 and stent graft second portion 108. Similarly, the arterial extension may have at least one stent hoop 426 expandable to support the arterial stent graft 424a. Alternatively, the stent hoop 426 can be a plurality of separate stent hoops connected to each other via the graft material for extension 424. As used herein, the term “separate” in relation to the stent hoops means that the hoops are not connected with connectors that are made of the same material as the hoop but via a different material. In the embodiment shown in FIG. 3, the stent hoop 426 is one stent being laser cut from a tube stock. It is preferable that the fenestrations or peripheral openings be disposed in the open space between the longitudinally spaced-apart sinusoidal like stent hoops. It is noted that the stent hoops are preferably sinusoidal (FIGS. 1 and 5) but do not have to be and therefore can be in an irregular zig-zag configuration (FIGS. 2 and 3).

By virtue of our design, we are able to account for variations in the biological anatomies where the renal arteries are oriented with respect to the abdominal aorta connected to the heart yet while maintaining a good seal between the artery wall and the main trunk section of the implant. That is, the main trunk section 108 can be deployed and then the first section 102 can be deployed thereafter such that a good seal is believed to be formed by the coupling of main trunk section 108 to the first section 102 at the junction where the aneurysm wall (“AW” in FIG. 4A) interfaces with the renal artery (indicated with the dashed circle as a sealing junction SJ in FIG. 4A). Where the AAA is presented as a suprarenal AAA as depicted in FIG. 4B, the seal junction SJ is believed to be form due to the coupling of the main section 108 and the first tubular section 102 to the main abdominal artery wall, indicated here in FIG. 4B.

Referring to FIG. 1, it can be seen that the first portion 102 is radially adjustable (indicated at R1) to the longitudinal axis L-L or with respect to the second portion 108 (indicated at R2) so that the first peripheral opening 104 on the first portion 102 can be aligned with the peripheral opening 128 on the second portion. The orientation of the peripheral openings can be determined using a suitable imaging technique, such as for example, a fluoroscopic imaging system via the use of radiopaque markers affixed to the first and second implant portions. While the orientation of opening 104 can be of any orientation, it is usually the case that first peripheral opening 104 (of first portion 102) is generally aligned to the second peripheral opening 128 on the second portion 108. Once the peripheral openings on the respective portions are aligned and arterial extension(s) 424 is inserted into these peripheral openings, tubular stent graft extensions 130 are provided for insertion into each of the two limbs 116, 118 to allow for fluid flow from the inlet opening 102b of the first portion 102 through the second portion 108 to the respective limbs 116, 118 of the implant and out through each of the tubular stent graft extension 130.

It is noted that while the peripheral openings are illustrated as circular openings formed on the circumference of the implant, other shapes and configurations can be utilized that are within the scope of the invention. For example, the peripheral opening may be in the shape of a truncated circular cone that tapers towards a smaller diameter as the conic peripheral opening extends away from the longitudinal axis. Alternatively, ridges or retention ribs may be provided on the circumference of the tapered cone to allow the ribs or ridges to retain the tapered conic peripheral opening in the inner surface of the branch artery without the use of a bridging stent-graft 424.

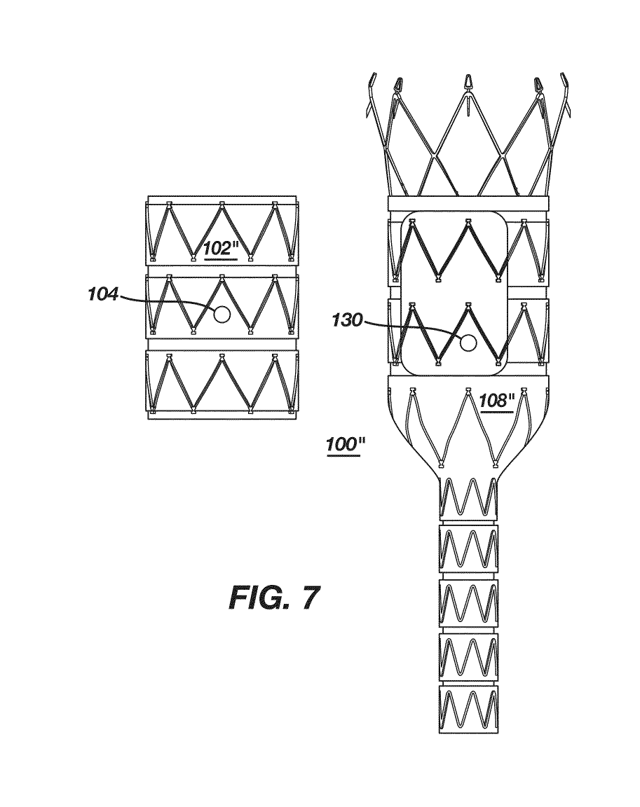

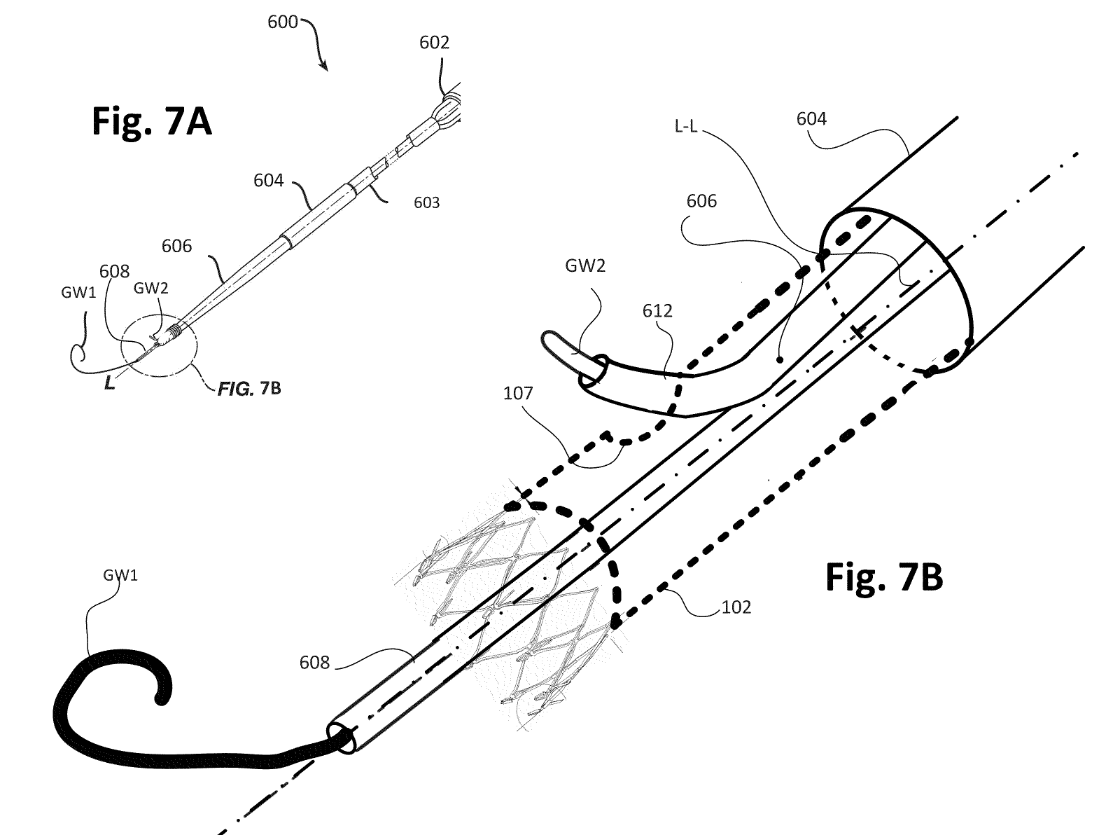

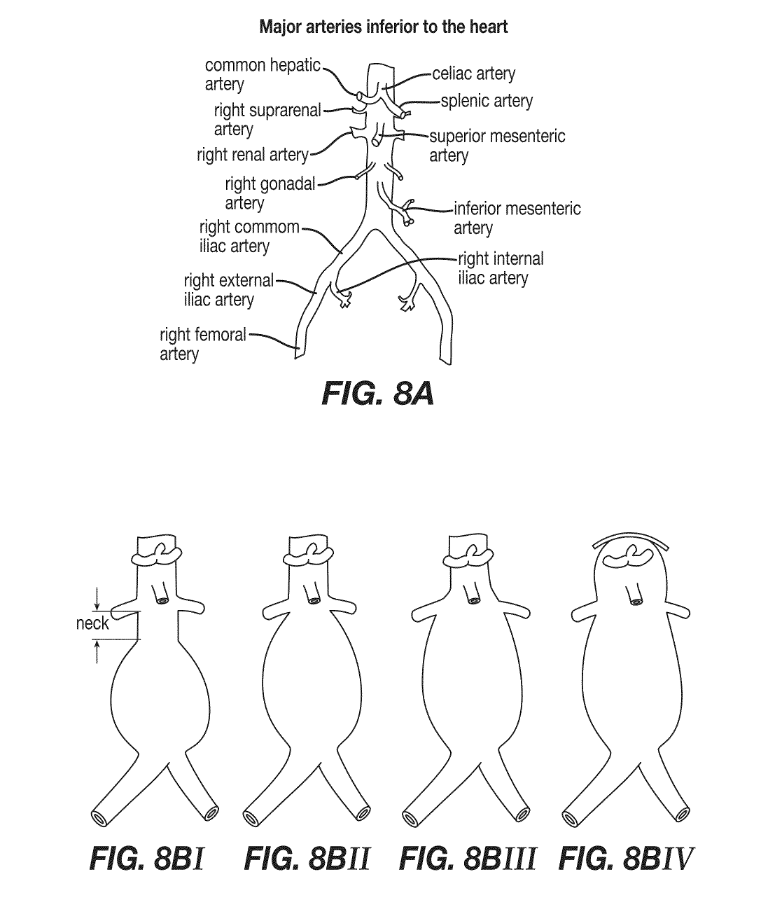

The peripheral openings or fenestrations can be configured with sutures 500 threaded on the circumference of the fenestration 104 (FIG. 1A) to provide for an initial small opening. Extra length 502 of the suture 500 can be provided at the end of the suture 500 to provide for slack to be built into the suture such that when the opening 104 is dilated, the slack 502 in the suture allows for enlargement of the fenestration to match a side branch artery of different diameters to the fenestration 104. The suture 500 can be configured with a predetermined slack length 502 to a lock stitch 504 to prevent over dilation of the peripheral opening 104. In addition to suture 500, reinforcement in the form of another type of suture can also be provided on the circumference of the peripheral opening 104. Radiopaque markers can be disposed on the circumference of the peripheral opening (or interwoven into the suture 500) so that the physician can visualize the actual size of the fenestration 107. The peripheral openings can be dilated to the intended size in-situ (in the native artery) by insertion of a suitable dilation balloon catheter guided to the fenestration via guidewire GW2 (FIG. 7B). Upon reaching the fenestration, the balloon can be inflated gradually while being monitored via the markers of the fenestration.

Referring to FIG. 7A, a delivery device 600 is shown in perspective view with a portion of the handle 602 proximal to the operator for manipulation by the operator is shown for brevity. Attention should be directed to the distal portion 603 which is provided with an outer sheath 604, a fenestration tube 606 that allows for an inner sheath 608 to pass through while being guided by a first guide wire GW1. The fenestration tube 606 also allows the implant (first or second portions) to be mounted so that the peripheral opening of the implant can be fixed to the fenestration tube 606 such that rotation of the fenestration tube 606 allows the peripheral opening of the graft implant to be aligned to the desired branch artery. In order to explain this unique aspect of the fenestration tube, reference is made to FIG. 7B, which is an enlarged perspective view of the distal end of the delivery device 600.

In FIG. 7B, the outer sheath 604 surrounds the outer surface of graft 102 while the inner surface of the graft 102 surrounds substantially the outer surface of the fenestration tube or sheath 606. The fenestration sheath 606 is substantially parallel to the outer surface of an inner sheath 608 which can pass through the outer sheath 604. To ensure proper alignment of the peripheral opening 107 of implant 102, the fenestration tube 606 is provided with a fenestration nub 612 on which the peripheral opening 107 is fitted over, all the while the implant being radially compressed in its pre-delivery profile. It should be noted here that while examples of the implant are shown in FIGS. 1-7 in its larger deployed profile (with the attendant large outside diameter conforming against the inner surface of an abdominal artery), the implant in its pre-delivery profile is compressed into the sheath 604 to a much smaller constrained profile (with a smaller outside diameter, as small as 14 French or less).

As is known in the art, the stent graft implant (e.g., implant 102) is moved to its intended location proximate the aneurysm by way of the inner sheath 608 following the first guide wire GW1. Once the implant 102 has arrived proximate the desired site, the outer sheath 604 can be pulled back (or the implant can be pushed out of the sheath 604) to expose the fenestration nub 612. This allows a second guide wire GW2 to be pushed out of the nub 612 via a lumen provided in the fenestration tube 606 (or in another lumen built into the inner sheath 608. Under an appropriate guidance technique (e.g., fluoroscopy), the second guide wire GW2 can be manipulated (via translation or rotation of fenestration tube 606 about its longitudinal axis L-L) so that guide wire GW2 can enter into an arterial branch (e.g., a renal artery RN1 or RN2 in FIG. 6). Insertion of the second guidewire GW2 into the arterial branch will ensure that the peripheral opening (e.g., 107) will adequately mate to the arterial branch. Where desired, the second guide wire can be utilized for insertion of the arterial extension or bridging stent. Thereafter, the other implant portion(s) can be inserted into the desired position along the first guidewire GW1 and deployed so that the other implant portion(s) can be coupled to the first implant portion.

Details of the handle and the procedures used for deployment of a similar AAA graft are shown and described in the Instruction for Use of the InCraft AAA implant (available in Europe), attached hereto the appendix. Where the AAA is presented other than an infrarenal AAA, the delivery device used for deployment can be via the device shown and described in U.S. Pat. No. 8,771,333, US Patent Application Publication Nos. US20070156224 and US20130085562, which are incorporated by reference as if set forth herein. It is noted that the examples provided are initially intended for AAAs, applications for other arterial sites with branching arteries can also be utilized such as, for example, in a thoracic aortic aneurysm or TAA where angulation of the artery may cause difficulty in forming a tight seal between the artery and the graft.

All of the stent hoops described herein are substantially tubular elements that may be formed utilizing any number of techniques and any number of materials. In the preferred exemplary embodiment, all of the stent hoops are formed from a nickel-titanium alloy Nitinol, shape set laser cut tubing.

The graft material utilized to cover all of the stent hoops may be made from any number of suitable biocompatible materials, including woven, knitted, sutured, extruded, or cast materials forming polyester, polytetrafluoroethylene, silicones, urethanes, and ultra-light weight polyethylene, such as that commercially available under the trade designation SPECTRA™. The materials may be porous or nonporous. Exemplary materials include a woven polyester fabric made from DACRON™ or other suitable PET-type polymers.

As noted above, the graft material is attached to each of the stent hoops. The graft material may be attached to the stent hoops in any number of suitable ways. In the exemplary embodiment, the graft material is attached to the stent hoops by sutures.

Depending on the stent hoops location, different types of suture knots may be utilized. Details of various embodiments of the suture knots for suture can be found in US Patent Application Publication No. US20110071614 filed on Sep. 24, 2009, which is hereby incorporated by reference as if set forth herein.

While the invention has been described in terms of particular variations and illustrative figures, those of ordinary skill in the art will recognize that the invention is not limited to the variations or figures described. For example, while examples are shown for AAA, these implants can also be utilized for thoracic aortic aneurysm (TAA), which may not require retention barbs for use in TAA. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. Therefore, to the extent there are variations of the invention, which are within the spirit of the disclosure or equivalent to the inventions found in the claims, it is the intent that this patent will cover those variations as well.

1. An endovascular implant comprising:
a first portion extending along a longitudinal axis, the first portion comprising a graft material defining a generally tubular graft extending from a first portion inlet opening to first portion outlet opening, the first portion including a first peripheral opening formed on a peripheral surface of the generally tubular graft to allow the first peripheral opening to communicate with the inlet and outlet of the first portion;
a second portion extending along the longitudinal axis, the second portion comprising a graft material defining a second portion inlet opening to a trunk section that extends along the longitudinal axis to a bifurcation section having two limbs with respective limb outlet openings, the second portion having a retention member coupled via a hoop structure to the inlet opening and two spaced apart peripheral openings formed through the peripheral surface of the trunk section to allow the spaced apart second portion's peripheral openings to communicate with the trunk inlet and limb outlets such that radial alignment of the first peripheral opening with one of the second peripheral openings allows fluid communication through the first peripheral opening and the aligned second peripheral opening, and
wherein the first peripheral opening of the first portion is formed through the graft material about the longitudinal axis of the first portion proximate the first end so that the first peripheral opening communicates with a mesenteric artery when the first and second portions are deployed together in an abdominal artery and the retention member of the second portion is disposed above both suprarenal and celiac arteries for the abdominal artery.

2. The endovascular implant of claim 1, in which the first peripheral opening comprises a scallop cut-out that extends from a periphery of the first portion to the outlet opening of the first portion.

3. The endovascular implant of claim 1, in which each of the first and second portions comprises a plurality of stent hoops spaced apart from each other along the longitudinal axis and attached to a graft material to define a composite implant, each of the stent hoops having a sinusoidal configuration disposed about the longitudinal axis with apices for a stent hoop being spaced apart along the longitudinal axis.

4. The endovascular implant of claim 3, in which one apex of one stent hoop is disposed between two apices of another stent hoop.

5. The endovascular implant of claim 3, in which the generally tubular graft comprises a material selected from a group consisting of nylon, ePTFE, PTFE, Dacron and combinations thereof.

6. The endovascular implant of claim 3 in which the plurality of stent hoops are disposed on a peripheral inside surface of the stent-graft.

7. (canceled)

8. The endovascular implant of claim 6, further comprising an additional first portion in which a second peripheral opening is formed through the graft material of the additional first portion about the longitudinal axis of the additional first portion so that the peripheral openings of the additional first portion are aligned with the second peripheral openings of the second portion to allow fluid communication with a renal artery when the implant is deployed in the abdominal artery.

9. The endovascular implant of claim 8, in which the first portion is radially adjustable with respect to the second portion so that the first peripheral opening on the first portion is generally aligned to the second peripheral opening on the second portion.

10. The endovascular implant of claim 9, in which a tubular stent graft extension is provided for insertion into each of the two limbs to allow for fluid flow from the inlet opening of the first portion through the second portion to the respective limbs of the implant and though each of the tubular stent graft extension.

11. The endovascular implant of claim 10, further comprising an arterial stent graft extension having a graft material in a generally tubular configuration with a generally circular opening at one end tapering towards a smaller second generally circular extension opening proximate another end, the arterial stent graft extension being configured for insertion into at least one of the peripheral openings of the first and second portions.

12. The endovascular implant of claim 11, in which the arterial stent graft extension includes at least one stent hoop expandable to support the arterial stent graft.
